(19) United States
(12) Patent Application Publication
(10) Pub. No.: US 2020/0078606 A1
(43) Pub. Date: Mar. 12, 2020

(54) HIGH-SPATIAL-RESOLUTION ULTRASONIC NEUROMODULATION METHOD AND SYSTEM

(71) Applicant: Xi'an Jiaotong University, Xi'an (CN)

(72) Inventors: Siyuan Zhang, Xi'an (CN); Zhiwei Cui, Xi'an (CN); Dapeng Li, Xi'an (CN); Tianqi Xu, Xi'an (CN); Shan Wu, Xi'an (CN); Mingxi Wan, Xi'an (CN)

(21) Appl. No.: 16/275,527

(22) Filed: Feb. 14, 2019

(30) Foreign Application Priority Data
Sep. 6, 2018 (CN) 201811036875.1

(51) Int. Cl. A61N 7/00

(52) U.S. Cl. CPC A61N 7/00; A61N 2007/0056; A61N 2007/0039; A61N 2007/0026

(57) ABSTRACT

A high-spatial-resolution ultrasonic neuromodulation method and system are provided. The method includes infusing a biological micro-nano material into the operating object by injection; aggregating the biological micro-nano material in the target region by a micro-nano manipulation method; and conducting ultrasonic neuromodulation on the target region by utilizing the ultrasound transducer and using an acoustic intensity between the first minimum acoustic intensity and the second minimum acoustic intensity. By using the method, an ultrasonic neuromodulation effect is generated only in a micro-nano material aggregation region by using a lower acoustic intensity, thereby reducing the threshold of ultrasonic neuromodulation, and greatly improving the spatial resolution of the ultrasonic neuromodulation.

### CROSS-REFERENCE TO RELATED APPLICATION

This application claims priority to Chinese application number 201811036875.1, filed on Sep. 6, 2018. The above-mentioned patent application is incorporated herein by reference in its entirety.

### TECHNICAL FIELD

The present invention relates to the field of neuromodulation, and more particularly, relates to an ultrasonic neuromodulation method and system.

### BACKGROUND

The research on brain science has attracted increasing attention from scientists and has become an extremely important research field now. Neuromodulation technology is one of the important branches of brain science research and treatment of related diseases, and ultrasonic neuromodulation is an emerging neuromodulation method in the last decade. Due to its non-invasive nature, ultrasonic neuromodulation has become a research focus in the field of neuromodulation. The spatial resolution of the ultrasonic neuromodulation is much higher than those of other non-invasive neuromodulation methods including magnetic stimulation. However, the spatial resolution thereof has yet to be improved as compared with other invasive neuromodulation methods such as deep brain stimulation and optogenetics.

The spatial resolution of existing ultrasonic neuromodulation is mainly determined by an ultrasound transducer. To achieve higher spatial resolution of the ultrasonic neuromodulation, a focused ultrasound transducer is generally used. A larger transducer diameter and higher ultrasound frequency can produce a smaller focal region of the focused ultrasound transducer, and thus the modulation resolution of the focused ultrasound transducer is higher. However, the target of ultrasonic neuromodulation is generally the brain, which means the ultrasonic wave needs to pass through the skull. A higher frequency ultrasound can result in the increasing ultrasonic attenuation when passing through the skull, and thus higher excitation sound pressure is required. This imposes extremely high requirements on the whole set of equipment, and thus it is difficult to achieve miniaturization and portability of ultrasonic-transducer driving equipment. The use of lower-frequency ultrasonic wave can properly solve the problem of the skull attenuating the ultrasonic wave, but for the lower-frequency ultrasonic wave, to achieve a modulation resolution on the grade of 1-2 mm, the diameter of the transducer for the lower-frequency ultrasonic wave is usually greater than 100 mm, which is not only difficult to operate, but also seriously affects the development of the transducer towards the wearable direction. Sonogenetics emerging in recent years can reduce the threshold of ultrasonic neuromodulation by expressing mechanically-sensitive membrane proteins on the cytomembrane of a nerve cell. Although using lower acoustic intensity only has a regulation effect on a transgenic-modified neural cell, this method is difficult to operate and requires transgenic operation, and thus is only suitable for the field of laboratory animal researches.

In view of the above, the existing high-resolution neuromodulation methods have higher invasive properties, or even need transgenic manipulation. Therefore, it is desirable to propose a novel micro-invasive or even non-invasive high-spatial-resolution ultrasonic neuromodulation method.

Therefore, it would be desirable to provide a high-resolution ultrasonic neuromodulation method and system, which can generate an ultrasonic neuromodulation effect only in a micro-nano material aggregation region by using a lower acoustic intensity, thereby greatly improving the spatial resolution of the traditional ultrasonic neuromodulation without the transgenic manipulation.

### SUMMARY

To achieve the above objective, the present invention provides the following solution, in one embodiment: A high-spatial-resolution ultrasonic neuromodulation method includes: acquiring a target region of an operating object; acquiring a first minimum acoustic intensity of an ultrasound transducer having a neuromodulation effect in a state where the target region has no biological micro-nano material; acquiring a second minimum acoustic intensity of the ultrasound transducer having a neuromodulation effect in a state where the target region has a biological micro-nano material; infusing a biological micro-nano material into the operating object by injection; aggregating the biological micro-nano material in the target region by a micro-nano manipulation method; and conducting ultrasonic neuromodulation on the target region by utilizing the ultrasound transducer and using an acoustic intensity between the first minimum acoustic intensity and the second minimum acoustic intensity.

In one aspect, the target region is a brain region or nervous tissue of the operating object to which the neuromodulation is targeted.

In another aspect, the biological micro-nano material is a liquid containing bubbles having a diameter of several nanometers to several micrometers, and the step specifically includes: using an ultrasonic microbubble contrast agent containing air or a high-density inert gas wrapped by materials such as a liposome, a polymer and various surfactants; and using a phase-change nano-ultrasound contrast agent in which a material such as a liposome, a polymer and various surfactants is used as a shell membrane material, and a material having phase change properties such as liquid fluorocarbons is used as a core.

In a further aspect, the second minimum acoustic intensity is smaller than the first minimum acoustic intensity, and that is, there is an effect of reducing the ultrasonic neuromodulation threshold after the biological micro-nano material is introduced into the target region.

In yet another aspect, the micro-nano manipulation method particularly includes: an acoustic micro-nano manipulation method using physical effects such as a standing wave sound field, an acoustic radiation force and acoustic eddy current; a method for conducting micro-nano manipulation of a magnetically-modified biological micro-nano material by using a magnetic field; and a method for conducting micro-nano manipulation of a micro-nano material which is modified in a targeted manner with respect to the target region in a molecular-biological target binding manner.

In another embodiment, the present invention further provides the following solution: A high-spatial-resolution ultrasonic neuromodulation system includes: a first acquisition module for acquiring a target region of an operating object; a second acquisition module for acquiring a first minimum acoustic intensity of an ultrasound transducer having a neuromodulation effect in a state where the target region has no biological micro-nano material; a third acquisition module for acquiring a second minimum acoustic intensity of the ultrasound transducer having a neuromodulation effect in a state where the target region has a biological micro-nano material; an infusion module for infusing a biological micro-nano material into the operating object by injection; a micro-nano manipulation module for aggregating the biological micro-nano material in the target region by a micro-nano manipulation method; and an ultrasonic neuromodulation module for conducting ultrasonic neuromodulation on the target region by utilizing the ultrasound transducer and using an acoustic intensity between the first minimum acoustic intensity and the second minimum acoustic intensity.

In one aspect, the target region is a brain region or nervous tissue of the operating object to which the neuromodulation is targeted.

In another aspect, the biological micro-nano material is a liquid containing bubbles having a diameter of several nanometers to several micrometers, and the step specifically includes: using an ultrasonic microbubble contrast agent containing air or a high-density inert gas wrapped by materials such as a liposome, a polymer and various surfactants; and using a phase-change nano-ultrasound contrast agent in which a material such as a liposome, a polymer and various surfactants is used as a shell membrane material, and a material having phase change properties such as liquid fluorocarbons is used as a core.

In a further aspect, the second minimum acoustic intensity is smaller than the first minimum acoustic intensity, and that is, there is an effect of reducing the ultrasonic neuromodulation threshold after the biological micro-nano material is introduced into the target region.

In yet another aspect, the micro-nano manipulation module particularly includes: a physical unit for conducting an acoustic micro-nano manipulation method using physical effects such as a standing wave sound field, an acoustic radiation force and acoustic eddy current; a first biological unit for a method of conducting micro-nano manipulation of a magnetically-modified biological micro-nano material by using a magnetic field; and a second biological unit for a method of conducting micro-nano manipulation of a micro-nano material which is modified in a targeted manner with respect to the target region in a molecular-biological target binding manner.

The embodiments of the present invention achieve the following technical effects: a high-resolution ultrasonic neuromodulation method is provided, which includes incorporating a biological micro-nano material capable of enhancing the biological effect of ultrasound, and aggregating the material in a small region of a nerve tissue by utilizing a micro-nano manipulation method such as acoustics, magnetic field manipulation or molecular biological target binding, where due to aggregation of the micro-nano material, this modulation enhancing effect only exists in a small range, so that an ultrasonic neuromodulation effect is generated only in a micro-nano material aggregation region by using a lower acoustic intensity, thereby reducing the threshold of ultrasonic neuromodulation, and greatly improving the spatial resolution of the ultrasonic neuromodulation.

### BRIEF DESCRIPTION OF THE DRAWINGS

Various additional features and advantages of the invention will become more apparent to those of ordinary skill in the art upon review of the following detailed description of one or more illustrative embodiments taken in conjunction with the accompanying drawings. The accompanying drawings, which are incorporated in and constitute a part of this specification, illustrate one or more embodiments of the invention and, together with the general description given above and the detailed description given below, explain the one or more embodiments of the invention.

FIG. 1 is a schematic flowchart of a high-spatial-resolution ultrasonic neuromodulation method according to one embodiment of the present invention.

FIG. 2 is a schematic structural diagram of a high-spatial-resolution ultrasonic neuromodulation system according to another embodiment of the present invention.

### DETAILED DESCRIPTION

The following clearly and completely describes the technical solutions in the embodiments of the present invention with reference to the accompanying drawings in the embodiments of the present invention. To make objectives, features, and advantages of the present invention clearer, the following describes embodiments of the present invention in more detail with reference to accompanying drawings and specific implementations.

The ultrasonic neuromodulation is a novel neuromodulation process. By applying ultrasonic stimulation to a targeted nerve region, the neural activity state of the targeted region is changed to achieve the purpose of neuromodulation. The ultrasonic neuromodulation has advantages such as being non-invasive and having stronger spatial resolution of modulation. The spatial resolution of traditional ultrasonic neuromodulation is mainly determined by the acoustic-field distribution characteristics of the ultrasound transducer, and in the acoustic field of the ultrasound transducer a region beyond the modulation threshold is a modulation region. Nano-scale or micro-scale air bubbles coated with lipids or other materials, phase-changed droplets, nano-scale and micron-scale air bubbles produced in other manners, and the like (hereinafter referred to as a micro-nano material for short) all can enhance the biological effects of ultrasound, so that the ultrasonic neuromodulation threshold can be reduced and the effect of the ultrasonic neuromodulation can be improved, and more importantly, this improvement effect only exists in a micron-scale range around the micro-nano material, and that is, this improvement effect only affects the nerve tissue in the micron-scale range around the micro-nano material. In contrast, the modulation spatial resolution achievable by an ultrasound transducer that is generally applied for the ultrasonic neuromodulation is on the grade of millimeter, which makes the spatial resolution of the ultrasonic neuromodulation be not only affected by the acoustic field distribution of the ultrasound transducer, but also associated with the spatial distribution of the micro-nano material. Based on this, the present invention provides a high-spatial-resolution ultrasonic neuromodulation method.

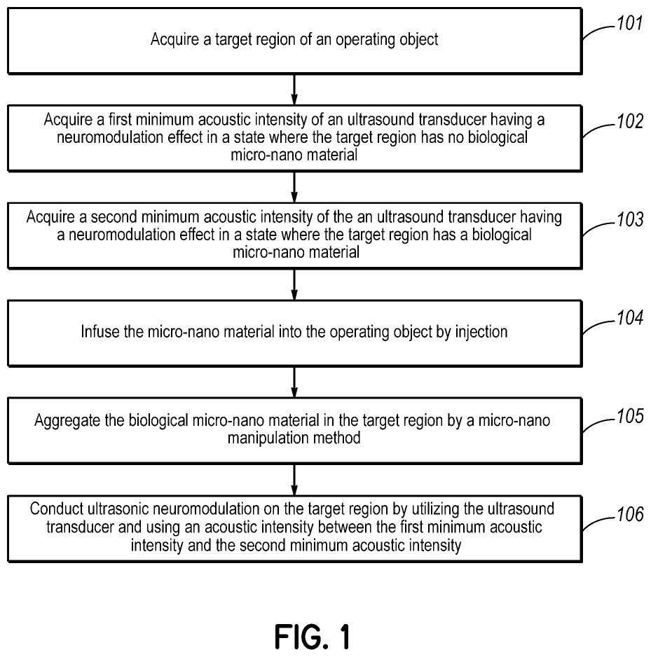

FIG. 1 is a flowchart of a high-spatial-resolution ultrasonic neuromodulation method according to an embodiment of the present invention. As shown in FIG. 1, a method for conducting high-spatial-resolution ultrasonic neuromodulation includes the following steps.
Step 101: a target region of an operating object is acquired; where the target region includes a region in which a target and a target setting range are modulated.
Step 102: a first minimum acoustic intensity of an ultrasound transducer having a neuromodulation effect in a state where the target region has no biological micro-nano material is acquired.
Step 103: a second minimum acoustic intensity of the ultrasound transducer having a neuromodulation effect in a state where the target region has a biological micro-nano material is acquired.

Taking the modulation in which the ultrasound stimulates the cerebral motor cortex to produce a motion response as an example, when there is no micro-nano material, the acoustic intensity of the ultrasonic stimulation is adjusted, and the modulation effect is observed, i.e., whether there is a motion response, where the threshold with the presence of the motion response is the first minimum acoustic intensity; the micro-nano material is added, and at this time, the micro-nano material cannot be aggregated in the target region as long as it is ensured that there is a micro-nano material in the target region, and then the acoustic intensity of the ultrasonic stimulation is adjusted again, and the motion response is observed, where the threshold with the presence of a response is the second minimum acoustic intensity.

Since the micro-nano material is not aggregated when the second threshold is confirmed, the modulation may exist in a wide range, but as long as it is ensured that the target region has the micro-nano material, it can be known that the minimum acoustic intensity required for ultrasonic neuromodulation can be generated when the micro-nano material exists at this position, when it is wanted to conduct high-resolution modulation, as long as the micro-nano material is aggregated in the target region and there is no micro-nano material at other positions, the high-resolution ultrasonic neuromodulation can be achieved at this point.
Step 104: the micro-nano material is infused into the operating object by injection, where the biological micro-nano material is a liquid containing bubbles having a diameter of several nanometers to several micrometers, and the step specifically includes: using an ultrasonic microbubble contrast agent containing air or a high-density inert gas wrapped by materials such as a liposome, a polymer and various surfactants; and using a phase-change nano-ultrasound contrast agent in which a material such as a liposome, a polymer and various surfactants is used as a shell membrane material, and a material having phase change properties such as liquid fluorocarbons is used as a core, where optionally, the second minimum acoustic intensity is smaller than the first minimum acoustic intensity, and that is, there is an effect of reducing the ultrasonic neuromodulation threshold after the biological micro-nano material is introduced into the target region.
Step 105: the biological micro-nano material is aggregated in the target region by a micro-nano manipulation method.
Step 106: ultrasonic neuromodulation is conducted on the target region by utilizing the ultrasound transducer and using an acoustic intensity between the first minimum acoustic intensity and the second minimum acoustic intensity.

In the step 105, the micro-nano manipulation method specifically includes: an acoustic micro-nano manipulation method using physical effects such as a standing wave sound field, an acoustic radiation force and acoustic eddy current; a method for conducting micro-nano manipulation of a magnetically-modified biological micro-nano material by using a magnetic field; and a method for conducting micro-nano manipulation of a micro-nano material which is modified in a targeted manner with respect to the target region in a molecular-biological target binding manner.

In view of the above, since the target regions to be modulated are different, and thus the ultrasound transducers as used are different, the confirmation of the first minimum acoustic intensity and the second minimum acoustic intensity requires previous pre-experiment acquisition. The target region is stimulated with ultrasonic waves of different sound intensities and it is observed whether there is a modulation effect to confirm the first minimum acoustic intensity. Thereafter the micro-nano material is injected (may not be aggregated in the target area, if it is ensured that the target region has the micro-nano material), and the target region is still stimulated through ultrasonic waves of different sound intensities and it is observed whether there is a modulation effect to confirm the second minimum acoustic intensity. Since the micro-nano material has an enhancement effect on the ultrasonic neuromodulation, the second minimum acoustic intensity is smaller than the first minimum acoustic intensity, and that is, there is an effect of reducing the ultrasonic neuromodulation threshold after the biological micro-nano material is introduced into the target region. At this point, when an acoustic intensity between the first minimum acoustic intensity and the second minimum acoustic intensity is used to perform ultrasonic stimulation in the target region, since the acoustic intensity is higher than the second minimum acoustic intensity, there is an effect of ultrasonic neuromodulation in a region having the micro-nano material, i.e., the target area, while there is no effect of ultrasonic neuromodulation in other regions having no micro-nano material since the acoustic intensity is lower than the first minimum acoustic intensity. In general, it is achieved that the modulation effect exists only in the target region where the micro-nano material is aggregated, and the modulation resolution is determined by the region where the micro-nano material is aggregated, thereby realizing a high spatial-resolution ultrasonic neuromodulation method by aggregating the biological micro-nano material in a small region.

In the ultrasonic neuromodulation method based on the enhancement by the micro-nano material as provided by the present invention, an ultrasonic neuromodulation effect with the lower threshold and the higher spatial resolution can be realized by adding infusion of the micro-nano material and the manipulation method for aggregating the micro-nano material based on the original non-invasive ultrasonic neuromodulation. The infusion of the micro-nano material and the manipulation method for aggregating the micro-nano material are all commonly-used operations in the field of medical ultrasound and micro-nano materials, which have extremely high security and extremely simple operation manner.

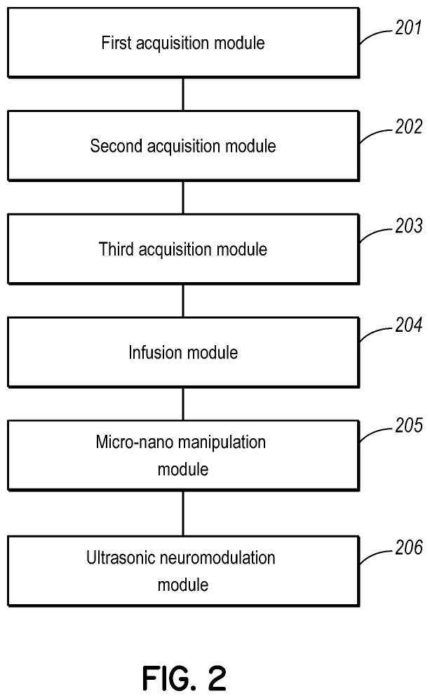

FIG. 2 is a structural diagram of a high-spatial-resolution ultrasonic neuromodulation system according to an embodiment of the present invention. As shown in FIG. 2, a high-spatial-resolution ultrasonic neuromodulation system includes: a first acquisition module 201 for acquiring a target region of an operating object; where the target region is a brain region or nervous tissue of the operating object to which the neuromodulation is targeted. a second acquisition module 202 for acquiring a first minimum acoustic intensity of an ultrasound transducer having a neuromodulation effect in a state where the target region has no biological micro-nano material; a third acquisition module 203 for acquiring a second minimum acoustic intensity of the ultrasound transducer having a neuromodulation effect in a state where the target region has a biological micro-nano material; where the second minimum acoustic intensity is smaller than the first minimum acoustic intensity, and that is, there is an effect of reducing the ultrasonic neuromodulation threshold after the biological micro-nano material is introduced into the target region, an infusion module 204 for infusing the micro-nano material into the operating object by injection, where the biological micro-nano material is a liquid containing bubbles having a diameter of several nanometers to several micrometers, and the step specifically includes: using an ultrasonic microbubble contrast agent containing air or a high-density inert gas wrapped by materials such as a liposome, a polymer and various surfactants; and using a phase-change nano-ultrasound contrast agent in which a material such as a liposome, a polymer and various surfactants is used as a shell membrane material, and a material having phase change properties such as liquid fluorocarbons is used as a core, a micro-nano manipulation module 205 for aggregating the biological micro-nano material in the target region by a micro-nano manipulation method; and an ultrasonic neuromodulation module 206 for conducting ultrasonic neuromodulation on the target region by utilizing the ultrasound transducer and using an acoustic intensity between the first minimum acoustic intensity and the second minimum acoustic intensity.

The micro-nano manipulation module 205 specifically includes: a physical unit for conducting an acoustic micro-nano manipulation method using physical effects such as a standing wave sound field, an acoustic radiation force and acoustic eddy current; a first biological unit for a method of conducting micro-nano manipulation of a magnetically-modified biological micro-nano material by using a magnetic field; and a second biological unit for a method of conducting micro-nano manipulation of a micro-nano material which is modified in a targeted manner with respect to the target region in a molecular-biological target binding manner.

### Embodiment 1

A brain region A was selected as the modulation target, and the brain region A has a range of a spherical region with a diameter of x mm.

For the ultrasonic neuromodulation of the brain region A, when the acoustic intensity is lower than Iₘₐₓ, the pure ultrasound effect is not enough to produce a stable modulation effect, and when the biological micro-nano material exists in the brain region A, a stable modulation effect can be generated when the acoustic intensity is higher than Iₘᵢₙ.

No limitation is applied to the method for aggregating the micro-nano material, and the manipulation aggregation of the micro-nano material can be conducted by using an acoustic standing wave method; or alternatively the micro-nano material is firstly modified to make it be magnetized, and that is the micro-nano material is manipulated and aggregated by using a magnetic field, and also for a specific brain region, the surface of the micro-nano material may be modified with an antigen-antibody targeted to a specific molecule in the target region, so that the micro-nano material is automatically aggregated in the target region by biochemical processes. Through the method, the micro-nano material can be aggregated in the spherical region with a diameter of x mm in the range of the brain region A.

Before the ultrasonic regulation, first the voltage should be excited by regulating the ultrasound transducer, so that the maximum acoustic intensity of the ultrasound transducer is Iₘₐₓ, and the distribution region where the acoustic intensity is between Iₘᵢₙ-Iₘₐₓ in the sound field is determined. It should be noted that, when the acoustic intensity is lower than Iₘₐₓ, the pure ultrasonic stimulation cannot achieve the purpose of ultrasonic neuromodulation; and when the acoustic intensity is higher than Iₘᵢₙ, the purpose of ultrasonic neuromodulation can be achieved through the enhancement effect of the micro-nano material, where in a general case, this region is larger than target region of modulation. Thereafter, the position and angle of the ultrasound transducer are adjusted by magnetic resonance imaging or other guiding manners, and the region where the acoustic field acoustic intensity of the transducer is between Iₘᵢₙ-Iₘₐₓ is used to cover the region where the micro-nano material is aggregated. At this point, when the ultrasound transducer emits an ultrasonic wave, due to the enhancement effect of the micro-nano material on the ultrasonic wave, the neuromodulation effect is generated only in the region where the micro-nano material is aggregated, i.e., the target region, while no neuromodulation is generated in other brain regions since the sound pressure is too low, thereby achieving ultrasonic neuromodulation with high spatial resolution.

Several examples are used for illustration of the principles and implementation methods of the present invention. The description of the embodiments is used to help illustrate the method and its core principles of the present invention. In addition, those skilled in the art can make various modifications in terms of specific embodiments and scope of application in accordance with the teachings of the present invention. In conclusion, the content of this specification shall not be construed as a limitation to the invention.

The embodiments described above are only descriptions of preferred embodiments of the present invention and are not intended to limit the scope of the present invention. Various variations and modifications can be made to the technical solution of the present invention by those of ordinary skill in the art, without departing from the design and spirit of the present invention. The variations and modifications should all fall within the claimed scope defined by the claims of the present invention.

What is claimed is:

1. A method for conducting high-spatial-resolution ultrasonic neuromodulation, comprising:
acquiring a target region of an operating object;
acquiring a first minimum acoustic intensity of an ultrasound transducer having a neuromodulation effect in a state where the target region has no biological micro-nano material;
acquiring a second minimum acoustic intensity of the ultrasound transducer having a neuromodulation effect in a state where the target region has a biological micro-nano material;
infusing a biological micro-nano material into the operating object by injection;
aggregating the biological micro-nano material in the target region by a micro-nano manipulation method; and
conducting ultrasonic neuromodulation on the target region by utilizing the ultrasound transducer and using an acoustic intensity between the first minimum acoustic intensity and the second minimum acoustic intensity.

2. The method of claim 1, wherein the target region is a brain region or nervous tissue of the operating object to which the neuromodulation is targeted.

3. The method of claim 1, wherein the biological micro-nano material is a liquid containing bubbles having a diameter of several nanometers to several micrometers, and the step of infusing the biological micro-nano material comprises:
using an ultrasonic microbubble contrast agent containing air or a high-density inert gas wrapped by materials such as a liposome, a polymer and various surfactants; and
using a phase-change nano-ultrasound contrast agent in which a material such as a liposome, a polymer and various surfactants is used as a shell membrane material, and a material having phase change properties such as liquid fluorocarbons is used as a core.

4. The method of claim 1, wherein the second minimum acoustic intensity is smaller than the first minimum acoustic intensity, and that is, there is an effect of reducing an ultrasonic neuromodulation threshold after the biological micro-nano material is introduced into the target region.

5. The method of claim 1, wherein the micro-nano manipulation method comprises:
an acoustic micro-nano manipulation method using physical effects such as a standing wave sound field, an acoustic radiation force and acoustic eddy current;
a method for conducting micro-nano manipulation of a magnetically-modified biological micro-nano material by using a magnetic field; and
a method for conducting micro-nano manipulation of a micro-nano material which is modified in a targeted manner with respect to the target region in a molecular-biological target binding manner.

6. A high-spatial-resolution ultrasonic neuromodulation system, comprising:
a first acquisition module for acquiring a target region of an operating object;
a second acquisition module for acquiring a first minimum acoustic intensity of an ultrasound transducer having a neuromodulation effect in a state where the target region has no biological micro-nano material;
a third acquisition module for acquiring a second minimum acoustic intensity of the ultrasound transducer having a neuromodulation effect in a state where the target region has a biological micro-nano material;
an infusion module for infusing a biological micro-nano material into the operating object by injection;
a micro-nano manipulation module for aggregating the biological micro-nano material in the target region by a micro-nano manipulation method; and
an ultrasonic neuromodulation module for conducting ultrasonic neuromodulation on the target region by utilizing the ultrasound transducer and using an acoustic intensity between the first minimum acoustic intensity and the second minimum acoustic intensity.

7. The system of claim 6, wherein the target region is a brain region or nervous tissue of the operating object to which the neuromodulation is targeted.

8. The system of claim 6, wherein the biological micro-nano material is a liquid containing bubbles having a diameter of several nanometers to several micrometers, and the infusion module operates by performing the following steps:
using an ultrasonic microbubble contrast agent containing air or a high-density inert gas wrapped by materials such as a liposome, a polymer and various surfactants; and
using a phase-change nano-ultrasound contrast agent in which a material such as a liposome, a polymer and various surfactants is used as a shell membrane material, and a material having phase change properties such as liquid fluorocarbons is used as a core.

9. The system of claim 6, wherein the second minimum acoustic intensity is smaller than the first minimum acoustic intensity, and that is, there is an effect of reducing an ultrasonic neuromodulation threshold after the biological micro-nano material is introduced into the target region.

10. The system of claim 6, wherein the micro-nano manipulation module particularly comprises:
a physical unit for conducting an acoustic micro-nano manipulation method using physical effects such as a standing wave sound field, an acoustic radiation force and acoustic eddy current;
a first biological unit for a method of conducting micro-nano manipulation of a magnetically-modified biological micro-nano material by using a magnetic field; and
a second biological unit for a method of conducting micro-nano manipulation of a micro-nano material which is modified in a targeted manner with respect to the target region in a molecular-biological target binding manner.
